(19) United States
(12) Patent Application Publication
(10) Pub. No.: US 2013/0073039 A1
(43) Pub. Date: Mar. 21, 2013

(54) PUPIL CENTERED FOVEA FOCUSED OPTICS ASSEMBLY FOR INTRAOCULAR LENS

(71) Applicant: Ram Srikanth Mirlay, Bangalore (IN)

(72) Inventor: Ram Srikanth Mirlay, Bangalore (IN)

(21) Appl. No.: 13/701,176

(22) PCT Filed: May 16, 2011
(86) PCT No.: PCT/IN2011/000340

§ 371 Date: Nov. 30, 2012

(30) Foreign Application Priority Data
May 31, 2010 (IN) 1489/CHE/2010

(51) Int. Cl. A61F 9/00

(52) U.S. Cl. CPC A61F 9/0017
USPC 623/6.38

(57) ABSTRACT

The embodiments herein provide a pupil centered and fovea focused optics assembly comprising a ring platform provided inside a capsular bag of a mammalian eye to support an intraocular lens. The optical center of the intraocular lens is decentered with respect to the geometric center of the intraocular lens to align the optical centre of the lens with a visual axis of a pupil of the mammalian eye to improve the visual quality and to prevent an aberration. A plane of the intraocular lens is turned and tilted through a preset angle to point an optic axis of the intraocular lens to the Fovea.

### CROSS-REFERENCE TO RELATED APPLICATIONS

This application is a National Phase Application of PCT International Application No. PCT/IN2011/000340, International Filing Date May 16, 2011, claiming priority of Indian Patent Application No. 1489/CHE/2010, filed May 31, 2010, which is hereby incorporated by reference.

### BACKGROUND OF THE INVENTION

1. Technical Field

The embodiments herein generally relate to the field of ophthalmology and particularly to an optical lens used in ophthalmology, for example, an implantable intraocular lens to be implanted in a mammalian eye. The embodiments herein more particularly relate to a pupil-centered and fovea-focused optics assembly for an implantable intraocular lens.

2. Description of the Related Art

Intraocular lenses (IOLs) are used in cataract eye surgery to replace the human/opacified mammalian (cataractous) lens for cataractous or refractive/or other reasons. A pupil of the mammalian eyes is not always centered to the optic axis of the eye. There is a normal, natural and common decentering of the pupil with respect to the optics of the eye. After surgical implementation of the IOL, it is often found that the center of the IOL optics does not coincide with the center of the pupil or with the visual axis. The optical axis of the IOL, which is the line from the geometric center of the IOL, and extended at normal or perpendicular to its surface, does not always fall on the Fovea Centralis, the most sensitive part of the retina. This results in a compromised sharpness of the vision (visual acuity or activity) and visual quality.

The normal human (mammalian) eye, seen as an optical device, has certain design defects, which limit its potential maximum vision. These are given as follows. First, the center of the pupil does not always exactly coincide with the center of the lens (natural or surgically implanted IOL), in other words, the pupil is de-centered from the optical center of the IOL, thereby causing an intrinsic reduction in the image quality on the retina. The de-centering can be up to 200 microns. Secondly, the optical axis of the human lens or the IOL when implanted does not fall exactly on Fovea Centralis, the most sensitive and ‘seeing’ (visual) point in the retina. It may be “off the axis” by a maximum of 7 degrees.

These two natural design defects exist in most of the human eyes in varying degrees. These are well-known medical facts for decades and are referred to as angle kappa, angle gamma, de-centered pupil, tilt of the natural lens etc.

When lenses opacify due to age or other reasons, cataract surgery is performed and a new artificial lens (IOL or Intra Ocular Lens) is implanted in the same place (Capsular BAG) as the original lens. Therefore, the IOL (prior art) too continues to inherit and cause the same pre-existing defects of mis-aligned optics (de-centered optics), i.e., the center of the IOL does not fall on/coincide with the center of the pupil and the optical axis of the IOL does not fall exactly on the Fovea, in most human/mammalian eyes.

At present, there is no system and method to align the geometric centre of the lens with the visual axis of the pupil and to focus (or point) the implanted lens optics towards the fovea, to improve the visual acuity and the visual quality. Hence, there is a need for a new system and method to align the optical centre of the implanted lens with the visual axis of the pupil and to tilt the implanted lens to focus towards the fovea.

### OBJECTS OF THE INVENTION

A primary objective of the embodiments herein is to develop an optics assembly to align the optical center of the implanted lens with the center (optical axis or visual axis) of the pupil to correct an aberration.

Another objective of the embodiments herein is to develop an optics assembly to tilt the implanted lens to focus (point) the lens axis on to the fovea to improve the sharpness and the quality of the vision towards the retinal capacity limit).

These and the other objects and advantages of this invention will be understood easily by studying the following specification with the accompanying drawings.

### SUMMARY OF THE INVENTION

Various embodiments herein provide a pupil-centered and fovea-focused optics assembly for an implantable intraocular lens. The assembly comprises a ring platform provided inside a capsular bag of a mammalian eye and an intraocular lens with an optical center and a geometric center mounted on the ring platform. The optical center of the intraocular lens is calculatedly de-centered with respect to the geometric center of the intraocular lens to align the optical centre of the lens with a visual axis of a pupil of the mammalian eye, to improve the visual quality.

According to one embodiment herein, a plane of the intraocular lens is turned and tilted through a preset angle to point an optic axis of the intraocular lens to the Fovea and the optical center of the intraocular lens is decentered from the geometric center of the intraocular lens to compensate for the decentered pupil.

According to one embodiment herein, the preset angle is calculated based on an overall diameter of the capsular bag, a thickness of the ring platform, a decentering direction and a decentering distance of the optical axis of the intraocular lens.

According to one embodiment herein, the overall diameter of the capsular bag, the thickness of the ring platform, the decentering direction and the decentering distance of the optical axis of the intraocular lens are calculated by performing a three dimensional scanning of the mammalian eye.

According to one embodimentherein, the intraocular lens is mounted on the ring platform through a plurality of haptics. The plurality of haptics holds the intraocular lens in a desired place and position.

According to one embodiment herein, the ring platform arranged inside the capsular bag supports the intraocular lens through the haptics and points the intraocular lens towards a fovea of the mammalian eye.

According to one embodiment herein, the haptics of the intraocular lens is fixed to a front part or a middle part or a back part of the ring platform. When an upper haptics of the intraocular lens is fixed to the front part of the ring platform, then a lower haptics is fixed to the back part of the ring platform.

According to one embodiment herein, the ring platform is provided with markings to identify a vertical meridian and a horizontal meridian. Similarly, the intraocular lens and the haptics are provided with the markings to identify a vertical meridian and a horizontal meridian.

According to one embodiment herein, the ring platform, the optic and the haptics are made of bio compatible material with the required rigidity, flexibility and optical qualities. The ring platform has an over-all diameter of 11 to 14 mm for humans and it can be varied for veterinary use depending on the species. It may be of hydrophobic or hydrophilic material. It may be coated with or stored/loaded with medications such as steroids, antibiotics, anti-glaucoma drugs, anti-degenerative drugs, anti-mitotic agents to reduce posterior capsular opacification, etc.

According to one embodiment herein, the size of the haptics is in the range of 0.5 mm-3.5 mm. The shape can be of any stable one but the number of haptics has to be 4 or more to provide stability against rolling.

According to one embodiment herein, the optic size is varied from 5 mm to 10 mm or more, based on the mammalian eye. The optic design too, can be of any functional type. It is also applicable to aspheric, multifocal, accommodative, and all types of intraocular lenses.

### BRIEF DESCRIPTION OF THE DRAWINGS

The other objects, features and advantages will occur to those skilled in the art from the following description of the preferred embodiment and the accompanying drawings in which:

FIG. 1 illustrates the front view of a pupil-centered intraocular lens, according to one embodiment herein.

FIGS. 2 and 2A illustrate the side view of a pupil-centered and fovea-focused intraocular lens, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE INVENTION

In the following detailed description, a reference is made to the accompanying drawings that form a part hereof, and in which the specific embodiments that may be practiced is shown by way of illustration. These embodiments herein are described in sufficient detail to enable those skilled in the art to practice the embodiments herein and it is to be understood that the logical, mechanical and other changes may be made without departing from the scope of the embodiments herein. The following detailed description is therefore not to be taken in a limiting sense.

A pupil-centered and fovea-focused optics assembly for an implantable intraocular lens comprises a ring platform provided to be placed inside a capsular bag of a mammalian eye and an intraocular lens with an optical center and a geometric center is mounted on the ring platform. The optical center of the intraocular lens is calculatedly de-centered with respect to the geometric center of the intraocular lens to align the optical centre of the lens with a visual axis of pupil of the mammalian eye, to improve the visual quality and to prevent an aberration.

According to one embodiment herein, a plane of the intraocular lens is turned and tilted through a preset angle to point an optic axis of the intraocular lens to the fovea and the optical center of the intraocular lens is decentered from the geometric center of the intraocular lens to compensate for the decentered pupil.

According to one embodiment herein, the preset angle is calculated based on an overall diameter of the capsular bag, a thickness of the ring platform, a decentering direction and a decentering distance of the optical axis of the intraocular lens.

According to one embodiment herein, the overall diameter of the capsular bag, the thickness of the ring platform, the decentering direction and the decentering distance of the optical axis of the intraocular lens are calculated by performing a three dimensional scanning measurement of the mammalian eye.

According to one embodiment herein, the intraocular lens is mounted on the ring platform through a plurality of haptics. The plurality of haptics holds the intraocular lens in a desired place.

According to one embodiment herein, the ring platform arranged inside the capsular bag supports the intraocular lens through the haptics and points the optic axis of the intraocular lens towards fovea centralis of the mammalian eye.

According to one embodiment herein, the haptics of the intraocular lens is fixed to a front part or a middle part or a back part of the ring platform. If upper haptics of the intraocular lens are fixed to the front part of the ring platform, then lower haptics are fixed to the back part of the ring platform.

According to one embodiment herein, the ring platform is provided with markings to identify a vertical meridian and a horizontal meridian. Similarly, the intraocular lens and the haptics are provided with the markings, to identify a vertical meridian and a horizontal meridian.

According to one embodiment herein, the ring platform, the optic and the haptics are made of bio-compatible material with the required rigidity, flexibility and optical qualities. The ring platform has an over-all diameter of 11 to 14 mm for humans and it is varied for veterinary use depending on the species. The thickness of the ring platform is varied from 1 mm to 2.5 mm. It is of hydrophobic or hydrophilic material. It is coated with or stored/loaded with medications such as steroids, antibiotics, anti-glaucoma drugs, anti-degenerative drugs, anti-mitotic agents, to reduce posterior capsular opacification, etc.

According to one embodiment herein, the size of the haptics is in the range of 0.5 mm-3.5 mm. The shape can be of any stable one but the number of haptics has to be 4 or more to provide stability against rolling.

According to one embodiment herein, the optic sizes are varied from 5 mm to 10 mm or more, based on the mammalian eye. The optic design too, can be of any functional type. It is also applicable to aspheric, multifocal, accommodative, and all types of intraocular lenses.

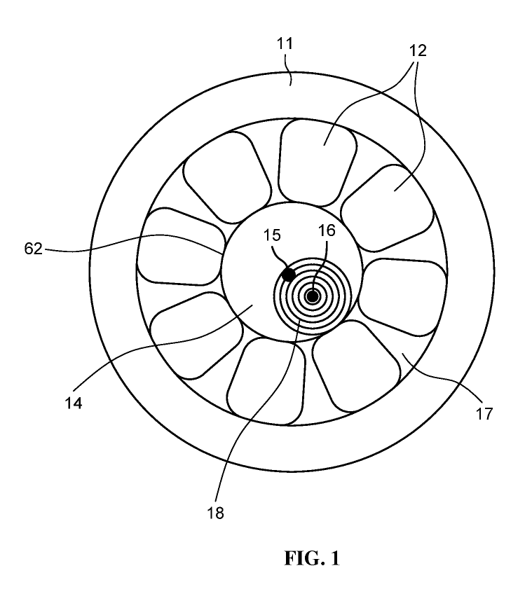

FIG. 1 illustrates the front view of the pupil-centered fovea-focused intraocular lens, according to one embodiment herein. A ring platform 11 is provided to be placed inside a capsular bag of a mammalian eye. An intraocular lens 19 is mounted on the ring platform 11. A plurality of haptics 12 are provided inside the ring platform 11 to exhibit improved strength and stability. The haptics 12 hold the intraocular lens 19 in its place. These haptics support structures 12 may be integrally formed with the intraocular lens 19 (as a one-piece lens) or separately manufactured and attached to the intraocular lens 19 (as a multi-piece lens). The intraocular lens 19 has a geometric center 15 and an optical center 16. The intraocular lens 19 has optical surfaces 14 and several refractive or diffractive zones 18. The distance between the geometric center 15 and the optical center 16 of the intraocular lens 19 is reduced and aligned with each other by decentering the optics. An empty space 17 exists between the adjacent haptics 12.

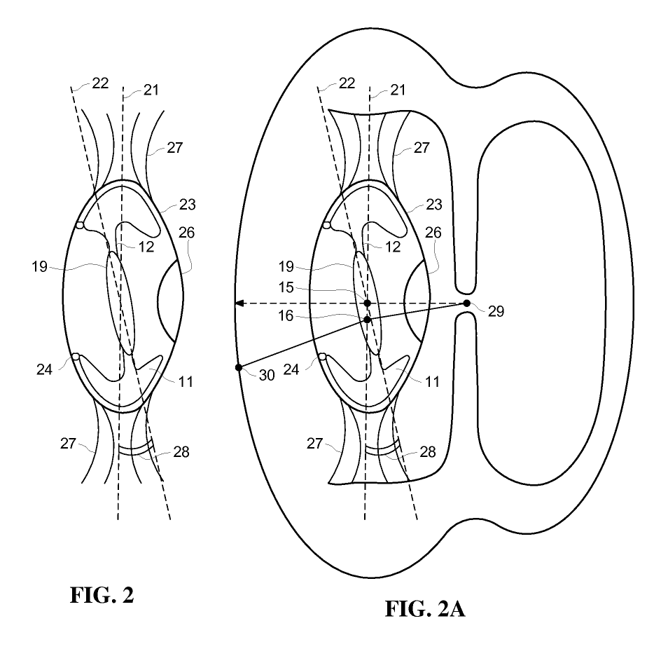

FIGS. 2 and 2A illustrate the side view of pupil-centered fovea-focused intraocular lens implanted in an eye, according to one embodiment herein. A capsular bag 23 and pupil 29, which are parts of a natural mammalian lens structure, is used provided to hold the intraocular lens 19 and a BAG-based foundation ring called ring platform for supporting the intraocular lens 19. Capsulorhexis 26 is the aperture formed by removing the lens capsule during cataract surgery. Zonules 27 are provided on both the poles. Zonules 27 are a series of fibers that pass from the ciliary body to the capsular bag of the lens at or near its equator thereby holding the intraocular lens 19 in its position and enabling the ciliary muscles to act upon them. The intraocular lens 19 and zonules 27 form a diaphragm dividing the eye into a small anterior area, which contains aqueous humor and a larger posterior area which contains vitreous humor. The zonules 27 form a ring that is roughly triangular in a meridional section. The zonules 27 are made up of fibers that are transparent and straight for the most part. The tension of these fibers varies with the state of contraction of the ciliary muscle and thus affects the convexity of the lens. A ring of square edge 24 is arranged next to the intraocular lens, supporting bag based platform ring 11 on the both the poles. The plane of the capsular bag 21 and plane of the intraocular lens 22 are not same. The capsular bag 23 is tilted by an angle of tilt and turn 28 to focus and align to the fovea 30.

The various embodiments herein provide an innovative design of the intraocular lens so that the geometric center of the lens is aligned to the visual axis, which lies in the center of the pupil to correct an aberration. Further a support is provided to hold and tilt the intraocular lens to focus its optical axis towards the fovea. The intraocular lens of the embodiments herein overcomes the natural visual aberration and increases the sharpness in vision and improves visual quality.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the embodiments herein with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

1-11. (canceled)

12. An optics assembly for a mammalian eye, the assembly comprising:
a ring platform disposed in capsular bag of the eye;
an intraocular lens mounted on the ring platform, with its optical center de-centered fromits geometric center and aligned with visual axis of pupil of the eye.

13. The optics assembly according to claim 12, wherein the plane of intraocular lens is disposed at an angle to the plane of the ring platform such that the optic axis of the intraocular lens points to fovea.

14. The optics assembly according to claim 1, wherein vertical and meridian markings are disposed on the ring platform.

15. The optics assembly according to claim 1, wherein the intraocular lens is mounted on the ring platform through plurality of haptics.

16. The optics assembly according to claim 1, wherein the haptics of the intraocular lens are fixed to a front, middle or back portion of the ring platform.

17. The optics assembly according to claim 1, wherein meridian markings are disposed on the intraocular lens.
